(19) United States
(12) Patent Application Publication
(10) Pub. No.: US 2003/0021785 A1
(43) Pub. Date: Jan. 30, 2003

(54) Use of rank antagonists to treat cancer

(76) Inventor: William C. Dougall, Seattle, WA (US)

Correspondence Address: IMMUNEX CORPORATION, LAW DEPARTMENT, 51 UNIVERSITY STREET, SEATTLE, WA, 98101

(21) Appl. No.: 10/166,232

(22) Filed: Jun. 5, 2002

Related U.S. Application Data
(60) Provisional application No. 60/296,670, filed on Jun. 6, 2001.

(51) Int. Cl. A61K 48/00; A61K 38/17; A61K 39/395

(52) U.S. Cl. 424/146.1; 514/12; 514/44

(57) ABSTRACT

Provided herein are methods of treating cancer by administering an effective amount of an agent that antagonizes the interaction between RANK and RANKL.

This patent application claims the benefit of priority under 35 U.S.C. §119 to U.S. Provisional Application Serial No. 60/296,670, the disclosure of which is incorporated herein by reference.

### TECHNICAL FIELD OF THE INVENTION

The present invention relates generally to the therapeutic use of antagonists of the RANK/RANKL interaction to treat cancer.

### BACKGROUND OF THE INVENTION

RANK (Receptor Activator of NF-κB) and its ligand RANKL) are a receptor/ligand pair that play an important role in immune responses and in bone metabolism. RANK and RANKL, both murine and human, have been cloned and characterized (see, for example, U.S. Pat. No. 6,017,729, WO 98/25958, EP 0 873 998, EP 0 911 342, U.S. Pat. No. 5,843,678, WO 98/46751 and WO 98/54201).

RANK, a Type I transmembrane protein, is a member of the TNF receptor superfamily (see, for example, U.S. Pat. No. 6,017,729). Full-length human RANK polypeptide has 616 amino acids. Human RANKL is a 317 amino acid protein of the tumor necrosis factor ligand family, and is a type II membrane protein lacking a signal peptide and having a short cytoplasmic domain and an extracellular region that binds specifically with RANK (see, for example, U.S. Pat. No. 6,017,729). RANKL has also been called “osteoprotegerin binding protein,” “osteoclastogenesis differentiation factor,” and “TRANCE” (see, for example, Kodaira et al., 1999; Yasuda et al., Proc. Natl. Acad. Sci. 95:3597 (1998); and Wong et al., J Biol Chem 273(43):28355-59 (1998)). RANKL binds not only to RANK, but also to a naturally occurring RANK decoy protein called osteoprotegerin (OPG), which is a member of the tumor necrosis factor receptor family (see, for example, U.S. Pat. No. 6,015,938 and WO 98/46751). OPG is a soluble molecule whose role in bone metabolism is reviewed in Hofbauer et al., J Bone Min Res 15(1):2-12 (2000). Further aspects of RANK/RANKL and OPG biology are discussed, for example, in Simonet et al., Cell 89:309-319 (1997); Kodaira et al., Gene 230:121-27 (1999); U.S. Pat. No. 5,843,678; and U.S. Pat. No. 6,015,938. In contrast to RANK, OPG also binds a second binding partner, which is known as “TNF-related apoptosis inducing ligand,” or “TRAIL.”

The RANK protein instigates intracellular events by interacting with various TNF Receptor Associated Factors (TRAFs) (see, for example, Galibert et al., J Biol Chem 273(51):34120-27 (1998); Darnay et al., J Biol Chem 273(32):20551-55 (1998); and Wong et al., 1998). The triggering of RANK, such as by its interaction with its receptor RANKL, activates TRAF-mediated intracellular events that result in the upregulation of the transcription factor NF-κB, a ubiquitous transcription factor that is extensively utilized in cells of the immune system. Signals mediated by the RANK/RANKL interaction are involved in stimulating the differentiation and function of osteoclasts, the cells responsible for bone resorption (see, for example, Lacey et al., Cell 93:165-76 (1998); Yasuda et al., 1998)). Accordingly, it has been proposed that osteoprotegerin or soluble forms of RANK could be used to inhibit osteoclast activity (see, for example, WO 98/46751, WO 99/58674, WO 01/16299 and Hofbauer et al., 2000). OPG or other antagonists of RANKL have been studied for their role in bone loss in a variety of systems, including hypercalcemia of cancer and osteolytic metastases (WO 98/46751; WO 01/03719; WO 01/16299; WO 01/17543; WO 01/03719; and Zhang et al., J Clin Invest 107:1235-44 (2001)). Several investigators have reported on the in vivo effects of RANK antagonists that are derived from the RANK protein (see, for example, U.S. Pat. No. 6,015,938 and WO 98/46751). Others have reported that administration of soluble RANK reduced bone destruction in mouse models of human disease (see Oyajobi et al., J Bone Min Res 15 (suppl. 1):S176, Abstract #1151 (September 2000); Oyajobi et al., Cancer Res 61:2572-78 (2001); Childs et al., Abstract, Orthopedic Research Society, San Francisco, 2001).

Some investigators have observed that certain cancer cells secrete a soluble form of RANKL that appears to contribute to hypercalcemia or to the establishment of malignant bone lesions (Nagai et al., Biochem Biophys Res Comm 269:532-536 (2000); and Zhang et al., 2001). Overproduction of parathyroid hormone-related protein also is believed to contribute to the hypercalcemia of cancer (see, for example, Rankin et al., Cancer (Suppl) 80(8):1564-71 (1997)). Hypercalcemia, a late complication of cancer, disrupts the body's ability to maintain a normal level of calcium, and can result in fatigue, calcium deposits in the kidneys, heart problems and neural dysfunction. Hypercalcemia occurs most frequently in patients with lung and breast cancer, and also is known to occur in patients with multiple myeloma, head and neck cancer, sarcoma, cancer of unknown primary origin, lymphoma, leukemia, melanoma, kidney cancer, and the gastrointestinal cancers, which includes esophageal, stomach, intestinal, colon and rectal cancers. The appearance of hypercalcemia has grave prognostic significance for cancer patients, with death following in one to three months for a majority of those in which it is present. Currently available hypocalcemic agents have little effect in decreasing the mortality rate among patients with hypercalcemia of malignancy. For this and other reasons, it is advantageous for patients with cancer to receive effective treatment during the early stages of disease before hypercalcemia has developed, and before metastasis has occurred.

### SUMMARY OF THE INVENTION

Provided herein are methods and compositions for using antagonists of the RANK/RANKL interaction to treat cancer.

Patients who will benefit from the treatments disclosed herein include those who have early stages of a type of cancer whose later stages are associated with hypercalcemia and/or bone metastases. In one embodiment of the invention, a RANK antagonist is administered to a patient having such a cancer prior to the development of hypercalcemia or metastasis to the bone. Additionally, the subject methods are used to treat patients suffering from various kinds of cancer whose later stages are not generally associated with hypercalcemia and/or metastasis to the bone.

The RANK antagonists used for this invention include an antibody that specifically binds RANK, an antibody that specifically binds RANKL, a small molecule that blocks the RANK/RANKL interaction or the synthesis of RANK or RANKL, an antisense oligonucleotide that blocks translation or transcription of RANK mRNA, or a soluble RANK polypeptide that is capable of binding RANKL. Soluble RANK proteins useful as RANK antagonists will comprise a RANKL-binding portion of the extracellular region of a RANK polypeptide.

### DETAILED DESCRIPTION OF THE INVENTION

The invention provides methods and compositions for treating primary cancer patients in early stages of disease. Such patients do not suffer from hypercalcemia, and may be treated before their cancer has metastasized. No bone lesions or osteolytic metastases are present in the patients to whom these methods are directed. Generally, the patient is a human, but the subject methods may be applied to any mammal, including domestic animals such as pets and farm animals. The subject methods involve administering to a patient in need thereof an amount of a RANK antagonist that is effective to inhibit tumor growth and/or metastasis. In the case of solid tumors, the subject treatments may result in tumor shrinkage. In the case of a hematologic cancer not characterized by solid tumor masses, the subject treatments may result in a reduction in the number of malignant cells detectable in the patient's blood. Moreover, the subject treatments may delay or prevent metastasis and/or hypercalcemia. As used herein, the terms “cancer” and “tumor” are used interchangeably to refer to any malignant disease, including solid tumors, blood-borne cancers and various hyperproliferative conditions.

RANK antagonists used for the subject therapeutic methods generally are proteins that are derived from the same species of animal as the patient. A “RANK agonist” is an agent that induces a biological activity associated with triggering RANK, such as inducing NF-κB activity. A “RANK antagonist,” as used herein, is an agent that blocks or reduces the interaction between RANK and RANKL, including agents that inhibit the synthesis of RANK or RANKL. RANK antagonists generally reduce one or more of the biological activities associated with triggering RANK, such as, for example, NF-κB activity, jun kinase activity, or stimulation of osteoclast differentiation. In certain embodiments, the RANK antagonist comprises a soluble RANK protein or an antibody against RANK or RANKL that inhibits or blocks the interaction between RANK and RANKL and that does not agonize RANK activity.

The treatments provided herein comprise administering to a non-hypercalcemic cancer patient an effective amount of a RANK antagonist, which in all instances described herein may be administered alone or in conjunction with other treatments such as resection surgery, radiation therapy, chemotherapy, monoclonal antibodies against tumor cell surface proteins, cytokines that have anti-tumor activity or agents that inhibit cytokines that promote tumor growth or survival. Cytokines suitable for concurrent administration with a RANK antagonist include GM-CSF and G-CSF. RANK antagonists may also be administered concurrently with a tumor vaccine. “Concurrent” administration encompasses simultaneous, alternating and sequential administration regimens.

The subject methods provide therapeutic treatments for patients who are in early stages of cancer and in whom hypercalcemia is not present. The term “hypercalcemia” refers here to a condition in which a cancer patient's serum calcium levels are above the normal range defined by the National Cancer Institute as 9.0 to 10.3 mg/dL (=4.5-5.2 mEq/L or 2.25-2.57 mmol/L) for men and 8.9 to 10.2 mg/dL (=4.4-5.1 mEq/L or 2.22-2.54 mmol/L) for women. Values measured for serum calcium levels may be corrected to account for hypoalbuminemia and/or acid-base status in accord with guidelines provided by the National Cancer Institute.

In one aspect of the invention, treatments are provided for patients having a type of cancer that has a predilection for metastasizing to the bone and in which hypercalcemia often appears during the late stages of disease. Treatment in accord with this invention is administered to such patients in early stages of their disease, prior to metastasis and prior to the appearance of hypercalcemia. Patients who will benefit from this method of treatment include those having cancer of the following types: lung; breast; head and neck; sarcoma; cancer of unknown primary origin; lymphoma; leukemia; melanoma; kidney; and gastrointestinal cancers, including esophageal, stomach, intestinal, colon, anal and rectal cancers.

In one embodiment of the invention, the methods described herein are used for treating patients who are in the early stages of prostate cancer and who are not hypercalcemic. Such patients are in stages A, B or C of prostate cancer, as determined according to the Jewett staging system. Using this staging system, stage A is a clinically undetectable tumor confined to the prostate gland and is an incidental finding at prostatic surgery; stage B is a tumor that is confined to the prostate gland; stage C is clinically localized to the periprostatic area but extending through the prostatic capsule and may involve seminal vesicles; stage D is metastatic disease. Alternatively, premetastatic prostate cancer patients may be identified by using the revised “TNM system,” which involves separate assessments of the primary tumor (T), lymph nodes (N) and metastases (M). The revised TNM system employs the same broad tumor stage (T stage) categories as the Jewett system, but includes subcategories of T stage, and PSA screening. Patients who are categorized as Stage I or stage II using this method are pre-metastatic, and are treated in accord with the present method.

Provided herein are methods of treating stage 0, I, II and III breast cancer in non-hypercalcemic patients by administering a RANK antagonist. For breast cancer, Stage 0 is called noninvasive carcinoma or carcinoma in situ, stages I and II are early stages in which the cancer has spread beyond the lobe or duct and invaded nearby tissue, stage III is locally advanced cancer, and stage IV is metastatic cancer.

The subject methods are useful for treating non-hypercalcemic patients with stage I and stage II renal or kidney cancer, including renal cell cancer and Wilm's tumor. For renal/kidney cancers staged in accord with NCI guidelines, stages I and II represent disease in which no cancer cells have penetrated the capsule that contains the kidney.

Provided herein are methods of treating stage 0, I, II and III lung cancer in non-hypercalcemic lung cancer patients by administering to a patient in need thereof a RANK antagonist. According to the currently used system for staging lung cancers, stages 0-III are non-metastastic, while stage IV is metastatic. Lung cancers include the non-small cell lung cancers, which are named for the type of cells found in the cancer and include squamous cell carcinoma (also called epidermoid carcinoma), adenocarcinoma, large cell carcinoma, adenosquamous carcinoma, and undifferentiated carcinoma. The subject methods for treating lung cancer includes treatment for the small cell lung cancers, including small cell carcinoma, mixed small cell/large cell carcinoma, combined small cell carcinoma (small cell lung cancer combined with neoplastic squamous and/or glandular components), and other neuroendocrine carcinomas of the lung, including the bronchial carcinoids, and the well-differentiated neuroendocrine carcinoma of the lung (also called malignant carcinoid, metastasizing bronchial adenoma, pleomorphic carcinoid, nonbenign carcinoid tumor, or atypical carcinoid).

In addition, the present methods of treatment are useful for treating myeloma-related syndromes, including plasma cell neoplasms such as plasmacytoma, monoclonal gammopathy of undetermined significance (MGUS), Waldenstrom macroglobulinemia and lymphoplasmacytic lymphoma. Such patients are not hypercalcemic when treatment is initiated.

Hematologic neoplasias and neoplastic-like conditions that can be treated with a RANK antagonist include but are not limited to Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymnphoma, hairy cell leukemia and lymphoplasmacytic leukemia); tumors of lymphocyte precursor cells, including B-cell acute lymphoblastic leukemia/lymphoma, and T-cell acute lymphoblastic leukemia/lymphoma; thymoma; tumors of the mature T and NK cells, including peripheral T-cell leukemias, adult T-cell leukemia/T-cell lymphomas and large granular lymphocytic leukemia; Langerhans cell histocytosis; myeloid neoplasias such as acute myelogenous leukemias, including AML with maturation, AML without differentiation, acute promyelocytic leukemia, acute myelomonocytic leukemia, and acute monocytic leukemias; myelodysplastic syndromes; and chronic myeloproliferative disorders, including chronic myelogenous leukemia.

The subject treatments also are useful for treating types of cancer that rarely or never metastasize to bone and in which hypercalcemia generally does not occur. These cancers maybbe treated prior to metastasis, and such cancers include but are not limited to: tumors of the central nervous system, such as brain tumors, including glioma, neuroblastoma, astrocytoma, medulloblastoma, ependymoma, and retinoblastoma; various solid tumors, including nasopharygeal cancer, basal cell carcinoma, pancreatic cancer, cancer of the bile duct, Kaposi's sarcoma, testicular cancer, uterine, vaginal or cervical cancers, ovarian cancer, primary liver cancer, or endometrial cancer; and tumors of the vascular system, including angiosarcomas, and hemagiopericytoma.

Antagonists of RANK suitable for use in the subject methods are characterized by their ability to inhibit or prevent biological manifestations of triggered RANK in a suitable assay, for example, in an assay that measures the biological activity of osteoclasts. Triggering of RANK, such as by contact with membrane-bound or soluble RANKL or with an agonistic anti-RANK antibody, instigates RANK-mediated cellular responses that can include the activation of transcription factor NF-κB, a ubiquitous transcription factor that is extensively utilized in cells of the immune system, and the activation of jun kinase (JNK; see, for example, Galibert et al., J. Biol. Chem. 273:34120-27 (1998)). Triggering RANK in osteoclast progenitor cells induces the progenitors to differentiate into mature osteoclasts. RANK activation also enhances the bone-resorption activity of mature osteoclasts.

The ability of a molecule to antagonize RANK and therefore be used in the subject methods can be readily determined, for example, in assays that measure the amount or activity of NF-κB in cells that express RANK, as described, for example, in U.S. Pat. No. 6,017,729, which is incorporated by reference herein in its entirety. In such an assay, cells that express RANK are used, such as 293/EBNA cells. 293/EBNA cells are a cell line that was derived by transfection of the 293 cell line with a gene encoding Epstein-Barr virus nuclear antigen-1. To perform such an assay, 293/EBNA cells or other RANK-expressing test cells are exposed to a RANK trigger in the presence or absence of a putative RANK antagonist. The RANK trigger can be cells that express RANKL or soluble RANKL or an antibody that agonizes RANK activity. After exposure to the putative antagonist, the amount or activity of NF-κB in the triggered test cells is measured. If the putative antagonist inhibited the triggering of RANK, the amount or activity of NF-κB will not be elevated in the triggered test cells. If less NF-κB is detected in test cells exposed to the putative RANK antagonist than in cells not exposed to the molecule, then the molecule is determined to be a RANK antagonist. Alternatively, JNK activation can serve as a measure of RANK activity for assessing potential RANK antagonists.

An exemplary nucleotide sequence encoding murine RANK is given in SEQ ID NO: 1, and an exemplary nucleotide sequence encoding human RANK is given in SEQ ID NO: 3; the corresponding full-length RANK polypeptides are shown, respectively, in SEQ ID NOS: 2 and 4. Human RANK protein has 616 amino acid residues, while murine RANK has 625 amino acids, each comprising an extracellular domain capable of binding RANKL, a transmembrane region and a cytoplasmic domain. The cytoplasmic domain of RANK is capable of binding TRAFs 1, 2, 3, 5 and 6. The extracellular domain of human RANK corresponds to amino acids 1-213 of SEQ ID NO: 4, and that of murine RANK to amino acids 1-214 of SEQ ID NO: 2. The human RANK protein has a signal sequence that may be cleaved after any amino acid between residues 24 and 33 of SEQ ID NO: 4, but which preferably is cleaved after amino acid 29. Murine RANK has a signal sequence that may be cleaved after any amino acid between residues 25 and 35 of SEQ ID NO: 2, but that preferably is cleaved between amino acids 30 and 31.

In one embodiment of the invention, patients in need thereof are treated by administering a RANK antagonist comprising a soluble RANK protein that is capable of binding RANKL that comprises all or a fragment of the extracellular domain of a RANK protein. The patient may be a human and the soluble RANK is derived from a human RANK polypeptide. Soluble RANK may comprise the signal peptide and the extracellular domain of the exemplary human or murine RANK polypeptides disclosed herein. Such polypeptides comprise, respectively, amino acids 1-213 of SEQ ID NO: 4 and amino acids 1-214 of SEQ ID NO: 2 or alternatively may comprise RANKL-binding fragments thereof. A useful RANK antagonist is one that comprises amino acids 30-213 of SEQ ID NO: 4. If desired, a RANK antagonist comprising amino acids 30-213 of SEQ ID NO: 4 may be fused to another protein that promotes dimerization.

RANK antagonists comprising a soluble RANK polypeptide may include other portions of RANK besides the extracellular domain but will not include the transmembrane region. The transmembrane regions of human and murine RANK are located, respectively, at amino acids 214-234 of SEQ ID NO: 4 and at amino acids 215-235 of SEQ ID NO: 2. Thus, soluble RANK antagonists suitable for the subject methods include proteins comprising a human RANK extracellular region fused directly to a RANK intracellular region, such as a protein comprising amino acids 30-213 and 235-625 of SEQ ID NO: 4 or RANKL-binding portions thereof.

The isolation of DNAs that encode human and murine RANK is described in U.S. Pat. No. 6,017,729. RANKL-binding variants and alleles of RANK can be obtained using the methods and reagents provided in U.S. Pat. No. 6,017,729. The isolation of an allelic variant of human RANK has been reported which differs only slightly from the amino acid sequence shown in SEQ ID NO: 4 (WO 98/54201). This variant of WO 98/54201, for example, has a valine instead of an alanine at the position corresponding to residue 192 of SEQ ID NO: 4, and an isoleucine instead of a serine at the position corresponding to residue number 513 of SEQ ID NO: 4. This RANK variant is capable of binding TRAFs and stimulating NF-κB and JNK. The human RANK proteins described in U.S. Pat. No. 6,017,729 or WO 98/54201 or any other RANKL-binding mutein or allelic variant of RANK may be used to derive soluble RANK proteins for use as antagonists in the subject invention. The ability of a RANK analog or mutein to be used to derive a soluble RANK for use as a RANK antagonist can be determined by testing the ability of the analogs or muteins to bind RANKL, for example as described in U.S. Pat. No. 6,017,729. Suitable assays for this purpose include, for example, cell based assays that measure NF-κB or JNK activity as described above, enzyme immunoassays or dot blots, assays that detect binding of labelled RANK to immobilized or cell-surface RANKL in the presence of increasing amounts of a putative antagonist that is expected to block RANK binding, or alternatively, assays that detect binding of labelled RANKL to immobilized or cell-surface RANK in the presence of a putative blocking agent. Such methods are well known in the art.

In one embodiment of the invention, soluble RANK polypeptides capable of binding RANKL are at least about 70% identical in amino acid sequence to the amino acid sequence of the extracellular region of native RANK protein as set forth in SEQ ID NOS: 2 or 4. In one embodiment, the soluble RANK polypeptides bind RANKL and are at least about 80% identical in amino acid sequence to the extracellular region of a RANK polypeptide as shown in SEQ ID NOS: 2 or 4. Generally, these soluble polypeptides are capable of binding RANKL and are at least about 90% identical to the extracellular region of the native form of RANK as shown in SEQ ID NOS: 2 or 4. Percent identity may be determined using a computer program, for example, the GAP computer program described by Devereux et al. (Nucl. Acids Res. 12:387 (1984)) and available from the University of Wisconsin Genetics Computer Group (UWGCG). For fragments derived from the RANK protein, the identity is calculated based on that portion of the RANK protein that is present in the fragment. When the murine and human RANK proteins of SEQ ID NOS: 2 and 4 are aligned as described here, they are found to be about 70% identical.

RANK antagonists useful for practicing the invention include soluble RANK polypeptides encoded by nucleic acid molecules that are capable of hybridizing under stringent conditions to a nucleic acid (or its complement) that encodes a RANKL-binding portion of a RANK extracellular region. Such RANK antagonists may further comprise a heterologous signal peptide or the Fc region of an immunoglobulin or some other moiety to facilitate synthesis, purification or clinical efficacy of the protein when used as a therapeutic agent. Selection of appropriate hybridization conditions is well-known in the art, and a number of options are described, for example, see Sambrook et al. (Molecular Cloning: A Laboratory Manual, 2nd ed. (Cold Spring Harbor Laboratory, Cold Spring Harbor, N.Y.; 1989); pages 9.50-9.57 and 11.45-11.57, which are hereby incorporated by reference). For probes longer than about 50 nucleotides in length, stringent conditions are achieved by hybridizing at a temperature that is 20-25° C. below the melting temperature (Tm), while for oligonucleotide probes (typically 14-40 nucleotides in length), stringent conditions generally entail hybridizing at a temperature 5-10° C. below the melting temperature (see Sambrook et al., page 11.45). For probes greater than about 14 nucleotides in length, Tm can be calculated with reasonable accuracy using the formula Tm (° C.)=81.5+16.6(log₁₀[Na⁺])+0.41(% G+C)−(600/N), where N is the number of bases in the hybrid duplex, and [Na⁺] is the concentration of sodium ions in the hybridization buffer ([Na⁺] for 1×SSC=0.165M) (see Sambrook et al., page 11.46). If formamide is added to a hybridization solution, the Tm and therefore the optimal hybridization temperature becomes reduced by about 0.63° C. for each 1% formamide (Sambrook et al. at page 9.51). When a target nucleic acid is fixed to a solid support, stringent hybridization conditions may be achieved, for example, by hybridizing in 6×SSC at 63° C., and washing in 3×SSC at 55° C. Alternatively, stringent conditions can be achieved by hybridizing in 6×SSC plus 50% formamide at 42° C., followed by washing at room temperature (about 22° C.) in 2×SSC, then washing in 0.2×SSC at 68° C.

In one embodiment, the nucleic acid molecule encoding a soluble RANK for use as a RANK antagonist in the subject invention will comprise nucleotides 91-642 of SEQ ID NO: 1 (murine RANK) or nucleotides 126-677 of SEQ ID NO: 3 (human RANK). The soluble RANK encoded by either of these nucleic acid molecules may correspond to any desired portion of a full-length RANK polypeptide so long as a sufficient amount of the RANK extracellular region is present to ensure binding to RANKL and the protein does not include the RANK transmembrane region. If desired, recombinant DNA techniques can be used to substitute a heterologous signal peptide for the native leader. A soluble RANK capable of binding RANKL may comprise a portion of human RANK having an amino terminus between amino acids 1 and 33 and continuing through amino acid 213 of SEQ ID NO: 4. RANKL-binding fragments comprising portions of such a protein are useful as RANK antagonists and can be identified by various binding assays, such as those described herein. Alternatively, unique restriction sites or PCR techniques that are known in the art can be used to prepare numerous truncated forms of RANK that can be expressed and analyzed for RANKL-binding activity.

Exemplary nucleic acids that encode RANKL-binding soluble RANK polypeptides suitable for use as RANK antagonists for the subject methods include:

(a) a nucleic acid molecule encoding a polypeptide comprising amino acids x to y of SEQ ID NO: 4, wherein x is selected from the group consisting of amino acids 1 to 33 of SEQ ID NO: 4, and y is selected from the group consisting of amino acids 196 to 213 of SEQ ID NO: 4;

(b) a nucleic acid molecule encoding a polypeptide comprising amino acids x to y of SEQ ID NO: 2, wherein x is selected from the group consisting of amino acids 1 to 35 of SEQ ID NO: 2, and y is selected from the group consisting of amino acids 197 to 214 of SEQ ID NO: 2; and

(c) a nucleic acid molecule capable of hybridizing under stringent conditions with a nucleic acid molecule of (a) or (b) or its complement, wherein the stringent conditions comprise hybridizing in 6×SSC at 63° C., and washing in 3×SSC at 55° C.

Soluble RANK proteins for use as antagonists within the scope of this invention include covalent or aggregative conjugates of the proteins or their fragments with other proteins or polypeptides, such as by synthesis in recombinant culture as N-terminal or C-terminal fusions. For example, the conjugated peptide may be a signal (or leader) polypeptide sequence at the N-terminal region of the protein which co-translationally or post-translationally directs transfer of the protein from its site of synthesis to its site of function inside or outside of the cell membrane or wall (e.g., the yeast α-factor leader). Protein fusions can comprise peptides added to facilitate purification or identification of RANK proteins and homologs (e.g., poly-His). The amino acid sequence of the inventive proteins can also be linked to an identification peptide such as that described by Hopp et al., Bio/Technology 6:1204 (1988) (FLAG™). Such a highly antigenic peptide provides an epitope reversibly bound by a specific monoclonal antibody, enabling rapid assay and facile purification of expressed recombinant protein. The sequence of Hopp et al. is also specifically cleaved by bovine mucosal enterokinase, allowing removal of the peptide from the purified protein.

Fusion proteins comprising a soluble RANK are highly desirable for use as RANK antagonists in the subject therapeutic methods. Such fusion proteins may comprise, for example, a moiety such as an immunoglobulin Fc domain, a FLAG™ tag, a poly(His) tag, which preferably has 6 His residues (SEQ ID NO: 6), a leucine zipper, polyethylene glycol or combinations thereof. An exemplary RANK:Fc fusion protein for use as a therapeutic agent is one having an amino acid sequence as shown in SEQ ID NO: 5, or having amino acids 30-443 of SEQ ID NO: 5.

Fusion proteins comprising RANKL-binding forms of soluble RANK suitable for use as described herein may be made using recombinant expression techniques. Such fusion proteins may form dimers or higher forms of multimers. Polymerized forms possess enhanced ability to inhibit RANK activity. Examples of fusion proteins that can polymerize include a RANK/Fc fusion protein, which can form dimers, and a fusion protein of a zipper moiety (i.e., a leucine zipper). Other useful fusion proteins may comprise various tags that are known in the art. Other antagonists of the interaction of RANK and RANKL (i.e., antibodies to RANKL, small molecules) also are useful in the subject therapeutic methods.

In one embodiment of the invention, the RANK antagonist is a fusion protein that comprises the amino acid sequence of a RANK linked to an immunoglobulin Fc region. If a human patient is being treated, the RANK and Fc moieties of the fusion protein preferably are derived from human sources. One Fc region that may be used for this purpose is one derived from a human IgG₁ immunoglobulin. Fragments of an Fc region may also be used, as can Fc muteins. For example, certain residues within the hinge region of an Fc region are critical for high affinity binding to FcγRI. Canfield and Morrison (J. Exp. Med. 173:1483 (1991)) reported that Leu₍₂₃₄₎ and Leu₍₂₃₅₎ were critical to high affinity binding of IgG₃ to FcγRI present on U937 cells. Similar results were obtained by Lund et al. (J. Immunol. 147:2657 (1991); Molecular Immunol. 29:53 (1991)). Such mutations, alone or in combination, can be made in an IgG₁ Fc region to decrease the affinity of IgG₁ for FcR. Depending on the portion of the Fc region used, a fusion protein may be expressed as a dimer, through formation of interchain disulfide bonds. If the fusion proteins are made with both heavy and light chains of an antibody, it is possible to form a protein oligomer with as many as four RANK regions. An exemplary RANK:Fc fusion protein for use as a RANK antagonist is that shown in SEQ ID NO: 5, which comprises the extracellular domain of a human RANK at amino acids 1-213 and an Fc region derived from a human IgG₁ immunoglobulin at amino acids 214-443. Amino acids 1-29 of SEQ ID NO: 5 correspond to a leader sequence that may be cleaved off after the protein is translated in mammalian cells, thus yielding a protein comprising amino acids 30-443 of SEQ ID NO: 5 for use as a RANK antagonist.

In another embodiment, RANK proteins used as a RANK antagonist further comprise an oligomerizing peptide such as a zipper domain. Leucine zippers were originally identified in several DNA-binding proteins and are present in the fos, jun and c-myc proteins (Landschulz et al., Science 240:1759 (1988)). “Zipper domain” is a term used to refer to a conserved peptide domain present in these (and other) proteins that is responsible for multimerization of the proteins. The zipper domain comprises a repetitive heptad repeat, with four or five leucine, isoleucine or valine residues interspersed with other amino acids. Examples of zipper domains are those found in the yeast transcription factor GCN4 and a heat-stable DNA-binding protein found in rat liver (C/EBP; Landschulz et al., Science 243:1681 (1989)). The products of the nuclear oncogenes fos and jun comprise zipper domains that preferentially form a heterodimer (O'Shea et al., Science 245:646 (1989); Turner and Tjian, Science 243:1689 (1989)). Zipper moieties useful for these purposes are described, for example, in U.S. Pat. No. 5,716,805.

In yet other embodiments of the invention, antagonists are used that have been designed to reduce the level of endogenous RANK or RANKL gene expression, e.g., using well-known antisense or ribozyme approaches to inhibit or prevent translation of RANK or RANKL mRNA transcripts; and triple helix approaches to inhibit transcription of RANK or RANKL genes. Techniques for the production and use of such molecules are well known to those of skill in the art.

Antisense RNA and DNA molecules useful as RANK antagonists can act to directly block the translation of mRNA by hybridizing to targeted endogenous mRNA thereby preventing translation. Alternatively, antisense oligonucleotides can be targeted to the RANK or RANKL genes to prevent their transcription blocking translation may be accomplished by using oligonucleotides (either DNA or RNA) that are complementary to RANK or RANKL mRNA, such as for example the anti-RANK antisense oligonucleotides described in U.S. Pat No. 6,171,860. Useful antisense oligonucleotides for this purpose include those that are complementary to the 5′ end of the mRNA, e.g., the 5′ untranslated sequence up to and including the AUG initiation codon. However, oligonucleotides complementary to the 5′- or 3′-non-translated, non-coding regions of the RANK or RANKL gene transcript, or to the coding regions, may be used. Antisense nucleic acids should be at least six nucleotides in length, and generally are oligonucleotides ranging from 6 to about 50 nucleotides in length. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleosides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane or hybridization-triggered cleavage agents or intercalating agents.

For delivery to cells expressing RANK or RANKL, antisense DNA or RNA can be injected directly into the tissue or cell derivation site, or modified antisense molecules designed to target the desired cells (e.g., antisense linked to peptides or antibodies that specifically bind receptors or antigens expressed on the target cell surface) can be administered systemically. In one approach, target cells are transfected with a recombinant DNA construct in which the antisense oligonucleotide is placed under the control of a strong pol III or pol II promoter. Such a vector can remain episomal or become chromosomally integrated, as long as it can be transcribed to produce the desired anti sense RNA. Vectors can be plasmid, viral, or others known in the art that are used for replication and expression in bacterial, yeast, insect or mammalian cells.

Ribozyme molecules designed to catalytically cleave RANK or RANKL mRNA transcripts can also be used to prevent translation of RANK or RANKL mRNA and expression of RANK or RANKL polypeptides. (See, e.g., WO 90/11364 or U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena thermophila (known as the IVS, or L-19 IVS RNA) and which has been extensively described (see, for example, WO 88/04300; Been and Cech, Cell, 47:207-216 (1986)). Ribozymes can be composed of modified oligonucleotides (e.g. for improved stability, targeting, etc.) and should be delivered to cells which express the RANK or RANKL polypeptide in vivo. One method of delivery that may be used involves using a DNA construct encoding the ribozyme under the control of a strong constitutive pol III or pol II promoter, so that transfected cells will produce sufficient quantities of the ribozyme to destroy endogenous RANK or RANKL messages and inhibit translation.

In yet other embodiments of the invention, the RANK antagonist used is an antibody that binds specifically with RANK or RANKL. Such antibodies include but are not limited to polyclonal antibodies, monoclonal antibodies (mABs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Antibodies that bind “specifically” are antibodies that bind their target, such as RANK or RANKL, via the antigen-binding sites of the antibody (as opposed to non-specific binding). Specifically binding antibodies will specifically recognize and bind a target RANK or RANKL polypeptide, such as those described herein, or subportions thereof, homologues, and variants thereof. Monoclonal antibodies to use as a RANK antagonist may be selected that are specific for epitopes present in human RANK or RANKL but not murine RANK or RANKL. Monoclonals that bind both mouse and human RANK or that bind both mouse and human RANKL also may be used as RANK antagonists for the subject therapeutic methods. Methods for obtaining monoclonal antibodies with a desired specificity are well known in the art, such as those described, for example, in U.S. Pat. No. 6,017,729. The RANK and RANKL polypeptides, fragments, variants and RANK fusion polypeptides as set forth herein can be employed as immunogens in producing antibodies specifically immunoreactive with RANK or RANKL. If the RANK antagonist is an anti-RANK antibody, the antibody when bound with the extracellular domain of RANK will not trigger RANK activity. An antagonistic anti-RANK antibody thus will not induce an increase in NF-κB activity in RANK-expressing cells.

RANK antagonists comprising a protein, such as purified soluble forms of RANK, antagonistic antibodies and homologs or analogs thereof are prepared by culturing suitable host/vector systems to express the recombinant translation products of the DNAs encoding the antagonist, which are then purified from culture media or cell extracts. A host cell that comprises an isolated nucleic acid of the invention, preferably operably linked to at least one expression control sequence, is a “recombinant host cell” and is said to be “transformed.”

To recombinantly express a RANK antagonist that is a polypeptide, isolated nucleic acids encoding the antagonist can be operably linked to an expression control sequence such as the pDC409 vector (Giri et al., EMBO J., 13:2821 (1990)) or the derivative pDC412 vector (Wiley et al., Immunity 3:673 (1995)). The pDC400 series vectors are useful for transient mammalian expression systems, such as CV-1 or 293 cells. Alternatively, the isolated nucleic acid can be linked to expression vectors such as pDC312, pDC316, or pDC317 vectors. The pDC300 series vectors all contain the SV40 origin of replication, the CMV promoter, the adenovirus tripartite leader, and the SV40 polyA and termination signals, and are useful for stable mammalian expression systems, such as CHO cells or their derivatives. Alternatively, nucleic acids encoding the antagonist may be expressed using a vector having an internal polyadenylation signal, such as those described in WO 01/27299. Other expression control sequences and cloning technologies can also be used to produce the polypeptide recombinantly, such as the pMT2 or pED expression vectors (Kaufman et al., Nucleic Acids Res. 19:4485-4490 (1991); and Pouwels et al., 1985, Cloning Vectors: A Laboratory Manual Elsevier, N.Y.) and the GATEWAY Vectors (Life Technologies; Rockville, Md.). In the GATEWAY system the isolated nucleic acid of the invention, flanked by attB sequences, can be recombined through an integrase reaction with a GATEWAY vector such as pDONR201 containing attP sequences. This provides an entry vector for the GATEWAY system containing the isolated nucleic acid of the invention. This entry vector can be further recombined with other suitably prepared expression control sequences, such as those of the pDC400 and pDC300 series described above. Many suitable expression control sequences are known in the art. General methods of expressing recombinant polypeptides are also described in R. Kaufman, Methods in Enzymology 185:537-566 (1990). As used herein, “operably linked” means that the nucleic acid of the invention and an expression control sequence are situated within a construct, vector, or cell in such a way that the polypeptide encoded by the nucleic acid is expressed when appropriate molecules (such as polymerases) are present. As one embodiment of the invention, at least one expression control sequence is operably linked to the nucleic acid of the invention in a recombinant host cell or progeny thereof, the nucleic acid and/or expression control sequence having been introduced into the host cell by transformation or transfection, for example, or by any other suitable method. As another embodiment of the invention, at least one expression control sequence is integrated into the genome of a recombinant host cell such that it is operably linked to a nucleic acid sequence encoding a polypeptide of the invention. In a further embodiment of the invention, at least one expression control sequence is operably linked to a nucleic acid of the invention through the action of a trans-acting factor such as a transcription factor, either in vitro or in a recombinant host cell.

A number of types of cells may act as suitable host cells for recombinant expression of polypeptides having RANK antagonist activity. Suitable mammalian host cells include, for example, the COS-7 line of monkey kidney cells (ATCC CRL 1651) (Gluzman et al., Cell 23:175 (1981)), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) as described by McMahan et al. (EMBO J 10:2821 (1991)), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, the polypeptide may be produced in lower eukaryotes such as yeast or in prokaryotes such as bacteria. Suitable yeast strains include Saccharomyces cerevisiae, Schizosaccharomyces pombe, Kluyveromyces strains, Candida spp., Pichia spp. or any yeast strain capable of expressing heterologous polypeptides. Potentially suitable bacterial strains include Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain a functional RANK antagonist. Such covalent attachments may be accomplished using known chemical or enzymatic methods.

The polypeptide may also be produced by operably linking the isolated nucleic acid of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988).

Cell-free translation systems may also be employed to produce polypeptides using RNAs derived from nucleic acid constructs disclosed herein.

The polypeptide of the invention may be prepared by culturing transformed host cells under culture conditions suitable to support expression of the recombinant polypeptide. The resulting expressed polypeptide may then be purified from such culture (i.e., from culture medium or cell extracts) using known purification processes, such as selective precipitation with various salts, gel filtration and ion exchange chromatography. The purification of the polypeptide may also include an affinity column containing agents that will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography using an antibody that specifically binds one or more epitopes of the RANK antagonist.

To harvest the polypeptide RANK antagonist, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. For example, a suitable affinity matrix can comprise a counter structure protein or lectin or antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Gel filtration chromatography also provides a means of purifying the inventive proteins.

Affinity chromatography is a particularly useful method of purifying RANK and homologs thereof. For example, a RANK expressed as a fusion protein comprising an immunoglobulin Fc region can be purified using Protein A or Protein G affinity chromatography. Moreover, a RANK protein comprising an oligomerizing zipper domain may be purified on a resin comprising an antibody specific to the oligomerizing zipper domain. Monoclonal antibodies against the RANK protein may also be useful in affinity chromatography purification, by utilizing methods that are well-known in the art. A ligand may also be used to prepare an affinity matrix for affinity purification of soluble RANK proteins or other RANK antagonists.

One or more reversed-phase high performance liquid chromatography (RP-HPLC) steps employing hydrophobic RP-HPLC media, e.g., silica gel having pendant methyl or other aliphatic groups, can be employed to further purify a RANK antagonist. Suitable methods include those analogous to the method disclosed by Urdal et al. (J. Chromatog. 296:171 (1984)). Some or all of the foregoing purification steps, in various combinations, can also be employed to provide a homogeneous recombinant protein.

Recombinant protein produced in bacterial culture is usually isolated by initial extraction from cell pellets, followed by one or more concentration, salting-out, aqueous ion exchange or size exclusion chromatography steps. Finally, high performance liquid chromatography (HPLC) can be employed for final purification steps. Microbial cells employed in expression of recombinant protein can be disrupted by any convenient method, including freeze-thaw cycling, sonication, mechanical disruption, or use of cell lysing agents. Fermentation of yeast which express the inventive protein as a secreted protein greatly simplifies purification.

Protein synthesized in recombinant culture is characterized by the presence of cell components, including proteins, in amounts and of a character which depend upon the purification steps taken to recover the inventive protein from the culture. These components ordinarily will be of yeast, prokaryotic or non-human higher eukaryotic origin and preferably are present in innocuous contaminant quantities, on the order of less than about 1% by weight. Further, recombinant cell culture enables the production of the inventive proteins free of other proteins which may be normally associated with the proteins as they are found in nature in the species of origin.

In practicing the subject therapeutic methods, the RANK antagonist is administered to a non-hypercalcemic cancer patient whose cancer has not metastasized to bone in an amount and at a frequency of administration that is effective to reach one or more of the following endpoints: a reduction in tumor burden; a stabilization of tumor burden; a slowing of the growth rate of the malignant cells; an increase in the length of time the patient remains disease free; and an increase in the length of time during which the cancer does not progress. In yet another aspect of the invention, the RANK antagonist is administered in an amount and at a frequency that is effective to reduce the amount of a surrogate marker that is associated with a particular type of cancer. Examples of such surrogate markers are serum HER2/neu in breast cancer and serum PSA for prostate cancer. The RANK antagonist may be administered to patients prior to or immediately following surgical removal of a solid tumor, or at any time post-surgery.

The duration of treatment will vary, but typically repeated doses will be administered over at least a period of two weeks or longer, or may be administered indefinitely. Several rounds of treatment may be given, alternating with periods of no treatment. If discontinued, treatment may be resumed if a relapse of the cancer should occur.

Treatment of cancer with a RANK antagonist may be administered concurrently with other treatments, and usually will be administered concurrently with chemotherapy or radiation treatment. In one example, the RANK antagonist is given concurrently with an agent that is effective against a variety of tumor types, such as Apo2 ligand/TRAIL or an anti-angiogenic agent such as an antibody against VEGF or an antibody against the EGF receptor. The RANK antagonist treatment also may be combined with other treatments that target specific kinds of cancer, such as for example, monoclonal antibodies targeted to tumor-specific antigens, or with other treatments used for particular kinds of cancer. For example, breast cancer may treated with a RANK antagonist administered concurrently with chemotherapy, hormone treatment, tamoxifen, raloxifene or agents that target HER2, such as an anti-HER2 antibody such as HERCEPTIN® (Genentech, Inc.), or any combination thereof. In another example, chronic lymphocytic leukemia or non-Hodgkin's lymphoma is treated with a combination of a RANK antagonist and the anti-CD20 monoclonal antibody RITUXIN® (Genentech, Inc.). The invention also contemplates the concurrent administration of RANK antagonists with various soluble cytokine receptors or cytokines or other drugs used for chemotherapy of cancer. “Concurrent administration” encompasses simultaneous or sequential treatment with the components of the combination, as well as regimens in which the drugs are alternated, or wherein one component is administered long-term and the other(s) are administered intermittently. Such other drugs include, for example, bisphosphonates used to restore bone loss in cancer patients, or the use of more than one RANK antagonist administered concurrently. Examples of other drugs to be administered concurrently include but are not limited to antivirals, antibiotics, analgesics, corticosteroids, antagonists of inflammatory cytokines, DMARDs, various systemic chemotherapy regimens and non-steroidal anti-inflammatories, such as, for example, COX I or COX II inhibitors.

One useful combination comprises the concurrent administration of a RANK antagonist and an antagonist of TNFα, which is a cytokine associated with inflammatory responses. TNFα inhibitors alone may be used to treat any of the conditions described herein, or may be used concurrently with a RANK antagonist. TNFα inhibitors that may be used include soluble proteins comprising the extracellular region of a TNFα receptor (TNFR), which may be derived from TNFR I or II or other TNFRs. One such TNFα inhibitor is etanercept, which is a dimer of two molecules of the extracellular portion of the p75 TNFα receptor, each molecule consisting of a 235 amino acid TNFR-derived polypeptide that is fused to a 232 amino acid Fc portion of human IgG₁. Etanercept is currently sold by Immunex Corporation under the trade name ENBREL,® and generally is administered 1-3 times per week by subcutaneous injection at a flat dose of 25 or 50 mg/dose or at a dose of 5-12 mg/m². Other suitable TNFα inhibitors include antibodies against TNFα, including humanized antibodies. An exemplary humanized antibody for coadministration with a RANK inhibitor is infliximab (sold by Centocor as REMICADE®), which is a chimeric IgG1κ monoclonal antibody. Other suitable anti-TNFα antibodies include the humanized antibodies D2E7 and CDP571, and the antibodies described in EP 0 516 785 B1, U.S. Pat. No. 5,656,272, and EP 0 492 448 A1. Additionally, TNFα may be inhibited by administering a TNFα-derived peptide that acts as a competitive inhibitor of TNFα (such as those described in U.S. Pat. No. 5,795,859 or U.S. Pat. No. 6,107,273), a TNFR-IgG fusion protein other than etanercept, such as one containing the extracellular portion of the p55 TNFα receptor, a soluble TNFR other than an IgG fusion protein, or other molecules that reduce endogenous TNFα levels, such as inhibitors of the TNFα converting enzyme (see e.g., U.S. Pat. No. 5,594,106), or small molecules such as pentoxifylline or thalidomide.

Similarly, inhibitors of the inflammatory cytokine IL-1 may be used alone to treat any of the cancers described above, or may be administered concurrently with a RANK antagonist. Suitable IL-1 inhibitors include, for example, receptor-binding peptide fragments of IL-1, antibodies directed against IL-1, including IL-1α or IL-1β or other IL-1 family members, antagonistic antibodies against IL-1 receptor type I, and recombinant proteins comprising all or portions of receptors for IL-1 or modified variants thereof, including genetically-modified muteins, multimeric forms and sustained-release formulations. Other useful IL-1 antagonists include IL-1ra polypeptides, IL-1β converting enzyme (ICE) inhibitors, IL-1 binding forms of type I IL-1 receptor and type II IL-1 receptor, and therapeutics known as IL-1 traps. IL-1ra polypeptides include the forms of IL-1ra described in U.S. Pat. No. 5,075,222 and modified forms and variants including those described in U.S. Pat. No. 5,922,573, WO 91/17184, WO 92 16221, and WO 96 09323. IL-1β converting enzyme (ICE) inhibitors include peptidyl and small molecule ICE inhibitors including those described in PCT patent applications WO 91/15577; WO93/05071; WO 93/09135; WO 93/14777 and WO 93/16710; and EP0547699. Non-peptidyl compounds include those described in WO 95/26958, U.S. Pat. No. 5,552,400, U.S. Pat. No. 6,121,266, and Dolle et al., J. Med. Chem. 39:2438-2440 (1996). Additional ICE inhibitors are described in U.S. Pat. Nos. 6,162,790, 6,204,261, 6,136,787, 6,103,711, 6,025,147, 6,008,217, 5,973,111, 5,874,424, 5,847,135, 5,843,904, 5,756,466, 5,656,627, 5,716,929. IL-1 binding forms of type I IL-1 receptor and type II IL-1 receptor are described in U.S. Pat. No. 4,968,607, U.S. Pat. No. 4,968,607, U.S. Pat. No. 5,081,228, U.S. Pat. No. Re 35,450, U.S. Pat. Nos. 5,319,071, and 5,350,683. IL-1 traps are described in WO 018932.

Further, suitable IL-1 antagonists encompass chimeric proteins that include portions of both an antibody molecule and an IL-1 antagonist molecule. Such chimeric molecules may form monomers, dimers or higher order multimers. Other suitable IL-1 antagonists include peptides derived from IL-1 that are capable of binding competitively to the IL-1 signaling receptor, IL-1 R type I.

Certain embodiments of the invention utilize type II IL-1 receptor in a form that binds IL-1 and particularly IL-1β, and blocks IL-1 signal transduction, thereby interrupting the proinflammatory and immunoregulatory effects of IL-1, and particularly that of IL-1β. U.S. Pat. No. 5,350,683 describes type II IL-1 receptor polypeptide. Certain forms of the type II IL-1 receptor polypeptide that may be used include truncated soluble fragments that retain the capability of binding IL-1 and particularly IL-1β. Soluble type II IL-1 receptor molecules useful as IL-1 antagonists include, for example, analogs or fragments of native type II IL-1 receptor that lack the transmembrane region of the native molecule, and that are capable of binding IL-1, particularly IL-1β.

Antagonists derived from type II IL-1 receptors (e.g. soluble forms that bind IL-1β) compete for IL-1 with IL-1 receptors on the cell surface, thus inhibiting IL-1 from binding to cells, thereby preventing it from manifesting its biological activities. Binding of soluble type II IL-1 receptor or fragments of IL-1 or IL-1β can be assayed using ELISA or any other convenient assay. If injected, the effective amount per adult dose of a soluble type II IL-1 receptor will range from 1-20 mg/m², and generally will be about 5-12 mg/m². Alternatively, a flat dose may be administered, whose amount will range from 5-100 mg/dose, or from 20-50 mg/dose.

Soluble type II IL-1 receptor polypeptides or fragments suitable in the practice of this invention may be fused with a second polypeptide to form a chimeric protein. In one embodiment of such a chimeric protein, the second polypeptide may promote the spontaneous formation by the chimeric protein of a dimer, trimer or higher order multimer that is capable of binding IL-1 molecule and preventing it from binding to a cell-bound receptor that promotes IL-1 signaling. Chimeric proteins used as antagonists may be proteins that contain portions of both an antibody molecule and a soluble type II IL-1 receptor.

For therapeutic use, a RANK antagonist is administered to an individual, including a human patient, for treatment in a manner appropriate to the indication. Systemic administration is generally appropriate for treating any type of cancer. the RANK/RANKL antagonist may be applied locally, which may be appropriate for skin cancers, though these patients may be treated systemically if desired. Means of local administration include, for example, local injection, or application of the antagonist admixed or polymerized with a slow-release matrix suitable for this purpose, many of which are known.

This invention additionally provides for the use of RANK antagonists and drugs to be concurrently administered with RANK antagonists in the manufacture of a medicament for the treatment of cancer. RANK antagonists and other drugs may be formulated into therapeutic compositions comprising an effective amount of the antagonist. In one embodiment of the invention, the therapeutic agent will be administered in the form of a pharmaceutical composition comprising a purified soluble protein having RANK antagonistic activity, in conjunction with physiologically acceptable carriers, excipients or diluents. Such carriers will be nontoxic to recipients at the dosages and concentrations employed. Inhibitors of the RANK/RANKL interaction for pharmaceutical compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Protein complexes with PEG can be made using known procedures, such as for example, those described in U.S. Pat. No. 5,849,860, U.S. Pat. No. 5,766,897 or other suitable methods. Suitable lipids for liposomal formulation include, without limitation, monoglycerides, diglycerides, cholesterol, sulfatides, lysolecithin, phospholipids, saponin, bile acids, and the like. Preparation of liposomal formulations is within the level of skill in the art, as disclosed, for example, in U.S. Pat. No. 4,235,871; U.S. Pat. No. 4,501,728; U.S. Pat. No. 4,837,028; U.S. Pat. No. 4,737,323; and U.S. Pat. No. 5,858,397. Such compositions will influence the physical state, solubility, stability, rate of in vivo release, and rate of in vivo clearance, and are thus chosen according to the intended application, so that the characteristics of the carrier will depend on the selected route of administration.

In one embodiment of the invention, sustained-release forms of RANK antagonists are used. Sustained-release forms suitable for use in the disclosed methods include, but are not limited to, soluble RANK polypeptides, and antagonistic anti-RANK or anti-RANKL antibodies that are encapsulated in a slowly-dissolving biocompatible polymer (such as the alginate microparticles described in U.S. Pat. No. 6,036,978), admixed with a slow-release polymer (including topically applied hydrogels), and/or incorporated into a biocompatible semi-permeable implant.

The amount of RANK antagonist administered per dose will vary depending on the antagonist being used and the mode of administration. If the antagonist is a soluble RANK and is administered by injection, the effective amount per adult dose will range from 0.5-20 mg/m², or from about 5-12 mg/m². Alternatively, a flat dose may be administered, whose amount may range from 5-100 mg/dose. Exemplary dose ranges for a flat dose to be administered by subcutaneous injection are 5-25 mg/dose, 25-50 mg/dose and 50-100 mg/dose. The chosen dose may be administered repeatedly, particularly for chronic conditions, or the amount per dose may be increased or decreased as treatment progresses. For pediatric patients (ages 4-17), a suitable regimen involves the subcutaneous injection of 0.4 mg/kg, up to a maximum dose of 25 mg to be administered one or more times per week. If an antibody against RANK or RANKL is used as the RANK antagonist, useful dose ranges include 0.1 to 20 mg/kg, 0.75 to 7.5 mg/kg and 1-10 mg/kg of body weight. Humanized antibodies are preferred, that is, antibodies in which only the antigen-binding portion of the antibody molecule is derived from a non-human source. Antibodies may be administered by injection, including intravenous infusion. Appropriate dosages can be determined in trials. The amount and frequency of administration will depend, of course, on such factors as the nature and severity of the indication being treated, the desired response, the condition of the patient, and so forth.

Ordinarily, the preparation of pharmaceutical compositions comprising a RANK antagonist entails combining the therapeutic protein with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with conspecific serum albumin are exemplary appropriate diluents. In certain embodiments, the product is formulated as a lyophilizate using appropriate excipient solutions (e.g., sterile water or sucrose solution) as diluents. One embodiment of the invention entails packaging a lyophilized RANK antagonist in dose unit form which when reconstituted will provide one to three doses per package.

The compounds of the present invention may be administered orally, parenterally, sublingually, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. Injection is a route of administration that may be used, including parenteral injection. Parenteral injections include subcutaneous injections, intraspinal, intrathecal, intraorbital, intravenous, intrarterial, intramuscular, intrasternal, and infusion techniques. Compositions comprising a RANK antagonist can be administered by bolus injection or continuous infusion. Routes of systemic administration that may be used include subcutaneous injection and intravenous drip.

In other embodiments of the invention, cells genetically modified to express a RANK antagonist are employed. For example, DNA encoding a soluble RANK or other protein with RANK antagonist activity is introduced into cells removed from the patient's body, and the cells thereafter returned to the patient. The DNA is introduced in a form that promotes expression of the antagonist in the recipient cells, that is, the coding regions are operably linked to appropriate regulatory elements for expression in the cells. The DNA may be introduced using a suitable vector, such as a retroviral or adenovirus vector, or encapsulated in liposomes. Suitable cells for this mode of drug administration include cells that will home to the affected tissue. In other similar embodiments, cell lines are modified to express the antagonist by introduction of DNA encoding the RANK antagonist, then the cells are introduced into the patient. Such cells may be transformed with DNA constructs that promote either stable or transient expression of the RANK antagonist. Alternatively, DNA encoding the antagonist may be introduced into the patient encapsulated in liposomes, which may be administered systemically or locally into the affected tissues.

Various animal models of the diseases to be treated are known in the art; accordingly, one can apply routine experimentation to determine optimal dosages and routes of administration of the RANK antagonist, first in an animal model and then in human patients. The specific dosing regimen for any particular patient will depend upon a variety of factors including the activity of the specific compound employed, the age, body weight, general health, sex, diet, time of administration, route of administration, rate of excretion, drug combination, and the severity of the patient's condition. It is expected that the patient's physician will adjust the dose and frequency of administration as needed to obtain optimal results.

### EXAMPLE

### Rank Expression on Human Cancer Cell Lines

Experiments were performed to demonstrate the expression of RANK on four different lines of human cancer cells, including two human breast cancer cell lines (MDA-MB-231 and MCF-7) and two human prostate cancer cell lines (PC-3 and DU145).

The cell lines described above were grown to about 75% confluency in 10 cm tissue culture plates. The media were decanted, then the cells were washed twice in PBS and lysed in 1 ml of lysis buffer (HNTG: 20 mM HEPES, pH 7.0, 150 mM NaCl, 0.1% Triton X-100, 10% glycerol). Clarified lysates were incubated with 5 mcg of a monoclonal antibody (mAb) specific for human RANK (clone M330or clone M331) at 4° C. for 1 hr. Immune complexes were purified by incubation with a mixture of ProteinA/ProteinG Sepharose beads (Pharmacia) and washed twice in HNTG and once in 50 mM Tris-HCl (pH 7.5). Purified immune complexes on beads were resuspended in SDS sample loading buffer, incubated at 100° C. to release the proteins from the beads and subjected to electrophoresis on 8-16% SDS/PAGE. After transfer of the fractionated proteins onto a membrane, RANK protein was revealed by western blotting using either a rat polyclonal raised against the entire human RANK cytoplasmic domain (GST-RANK CYTO) or a mAb specific for a peptide derived from human RANK (9A725). RANK was detected in fractionated proteins derived from all four of the tested cell lines.

What is claimed is:

1. A method of treating a cancer in a human patient who is not hypercalcemic and whose cancer has not metastasized to bone, the method comprising administering a RANK antagonist to said patient, wherein said patient has a serum calcium level between 9.0 to 10.3 mg/dL if the patient is a man and between 8.9 to 10.2 mg/dL if the patient is a woman, and further wherein the RANK antagonist is administered in an amount and at a frequency effective to reach an endpoint selected from the group consisting of reducing the tumor burden in said patient and slowing the growth rate of malignant cells in said patient.

2. A method according to claim 1, wherein the RANK antagonist is selected from the group consisting of an antibody that specifically binds RANK, an antibody that specifically binds RANKL, osteoprotegerin and an antisense oligonucleotide that blocks translation or transcription of RANK mRNA.

3. A method according to claim 1, wherein the RANK antagonist is a soluble RANK polypeptide that is capable of binding RANKL.

4. The method of claim 3, wherein the soluble RANK polypeptide is encoded by a nucleic acid molecule selected from the group consisting of:
(a) a nucleic acid molecule encoding a polypeptide comprising amino acids x to y of SEQ ID NO: 4, wherein x is selected from the group consisting of amino acids 1 to 33 of SEQ ID NO: 4, and y is selected from the group consisting of amino acids 196 to 213 of SEQ ID NO: 4;
(b) a nucleic acid molecule encoding a polypeptide comprising amino acids x to y of SEQ ID NO: 2, wherein x is selected from the group consisting of amino acids 1 to 35 of SEQ ID NO: 2, and y is selected from the group consisting of amino acids 197 to 214 of SEQ ID NO: 2; and
(c) a nucleic acid molecule capable of hybridizing under stringent conditions with a nucleic acid molecule of (a) or (b) or its complement, wherein the stringent conditions comprise hybridizing in 6×SSC at 63° C., and washing in 3×SSC at 55° C.

5. A method according to claim 4, wherein the soluble RANK polypeptide further comprises a moiety selected from the group consisting of an immunoglobulin Fc domain, a FLAG™ tag, a peptide comprising at least about 6 His residues, a leucine zipper, polyethylene glycol and combinations thereof.

6. A method according to claim 5, wherein the soluble RANK polypeptide comprises amino acids 30-443 of SEQ ID NO: 5.

7. A method of according to claim 1, wherein the patient has a cancer selected from the group consisting of plasmacytoma, monoclonal gammopathy of undetermined significance (MGUS), and Waldenstrom macroglobulinemia.

8. The method of claim 1, wherein the patient has a cancer selected from the group consisting of lung cancer, breast cancer, melanoma, sarcoma, prostate cancer, head and neck cancer, cancer of unknown primary origin, lymphoma, leukemia, kidney cancer, and gastrointestinal cancer.

9. The method of claim 1, wherein the patient has a cancer selected from the group consisting of brain tumor; glioma; neuroblastoma; astrocytoma; medulloblastoma; ependymoma; retinoblastoma; nasopharygeal cancer; basal cell carcinoma; pancreatic cancer; cancer of the bile duct; Kaposi's sarcoma; thymoma; testicular cancer; uterine cancer; vaginal cancer; cervical cancer; ovarian cancer; liver cancer; endometrial cancer; and hemagiopericytoma.

10. The method of claim 1, wherein the patient has a cancer selected from the group consisting of Hodgkin's lymphoma; non-Hodgkin's lymphoma; B-cell acute lymphoblastic leukemia/lymphoma; T-cell acute lymphoblastic leukemia/lymphoma; peripheral T-cell leukemia, adult T-cell leukemia/T-cell lymphoma; NK cell tumor; large granular lymphocytic leukemia; Langerhans cell histiocytosis; myeloid neoplasia; acute myelogenous leukemia; acute promyelocytic leukemia; acute myelomonocytic leukemia; acute monocytic leukemia; a myelodysplastic syndrome; and a chronic myeloproliferative disorder.

11. A method of treating a cancer in a human patient who is not hypercalcemic and whose cancer has not metastasized to bone, the method comprising administering a RANK antagonist to said patient, wherein said patient has a serum calcium level between 9.0 to 10.3 mg/dL if the patient is a man and between 8.9 to 10.2 mg/dL if the patient is a woman, and further wherein the RANK antagonist is administered in an amount and at a frequency effective to stabilize the tumor burden in said patient.

12. A method according to claim 11, wherein the RANK antagonist is selected from the group consisting of an antibody that specifically binds RANK, an antibody that specifically binds RANKL, osteoprotegerin and an antisense oligonucleotide that blocks translation or transcription of RANK mRNA.

13. A method according to claim 11, wherein the RANK antagonist is a soluble RANK polypeptide that is capable of binding RANKL.

14. The method of claim 13, wherein the soluble RANK polypeptide is encoded by a nucleic acid molecule selected from the group consisting of:
(a) a nucleic acid molecule encoding a polypeptide comprising amino acids x to y of SEQ ID NO: 4, wherein x is selected from the group consisting of amino acids 1 to 33 of SEQ ID NO: 4, and y is selected from the group consisting of amino acids 196 to 213 of SEQ ID NO: 4;
(b) a nucleic acid molecule encoding a polypeptide comprising amino acids x to y of SEQ ID NO: 2, wherein x is selected from the group consisting of amino acids 1 to 35 of SEQ ID NO: 2, and y is selected from the group consisting of amino acids 197 to 214 of SEQ ID NO: 2; and
(c) a nucleic acid molecule capable of hybridizing under stringent conditions with a nucleic acid molecule of (a) or (b) or its complement, wherein the stringent conditions comprise hybridizing in 6×SSC at 63° C., and washing in 3×SSC at 55° C.

15. A method according to claim 14, wherein the soluble RANK polypeptide further comprises a moiety selected from the group consisting of an immunoglobulin Fc domain, a FLAG™ tag, a peptide comprising at least about 6 His residues, a leucine zipper, polyethylene glycol and combinations thereof.

16. A method according to claim 15, wherein the soluble RANK polypeptide comprises amino acids 30-443 of SEQ ID NO: 5.

17. A method of according to claim 11, wherein the patient has a cancer selected from the group consisting of plasmacytoma, monoclonal gammopathy of undetermined significance (MGUS), and Waldenstrom macroglobulinemia.

18. The method of claim 11, wherein the patient has a cancer selected from the group consisting of lung cancer, breast cancer, melanoma, sarcoma, prostate cancer, head and neck cancer, cancer of unknown primary origin, lymphoma, leukemia, kidney cancer, and gastrointestinal cancer.

19. The method of claim 11, wherein the patient has a cancer selected from the group consisting of brain tumor; glioma; neuroblastoma; astrocytoma; medulloblastoma; ependymoma; retinoblastoma; nasopharygeal cancer; basal cell carcinoma; pancreatic cancer; cancer of the bile duct; Kaposi's sarcoma; thymoma; testicular cancer; uterine cancer; vaginal cancer; cervical cancer; ovarian cancer; liver cancer; endometrial cancer; and hemagiopericytoma.

20. The method of claim 11, wherein the patient has a cancer selected from the group consisting of Hodgkin's lymphoma; non-Hodgkin's lymphoma; B-cell acute lymphoblastic leukemia/lymphoma; T-cell acute lymphoblastic leukemia/lymphoma; peripheral T-cell leukemia, adult T-cell leukemia/T-cell lymphoma; NK cell tumor; large granular lymphocytic leukemia; Langerhans cell histiocytosis; myeloid neoplasia; acute myelogenous leukemia; acute promyelocytic leukemia; acute myelomonocytic leukemia; acute monocytic leukemia; a myelodysplastic syndrome; and a chronic myeloproliferative disorder.

21. A method of treating a cancer in a human patient who is not hypercalcemic and whose cancer has not metastasized to bone, the method comprising administering a RANK antagonist to said patient, wherein said patient has a serum calcium level between 9.0 to 10.3 mg/dL if the patient is a man and between 8.9 to 10.2 mg/dL if the patient is a woman, wherein the RANK antagonist is administered in an amount and at a frequency effective to increase in the length of time the patient remains disease free or to increase in the length of time during which the cancer does not progress.

22. A method according to claim 21, wherein the RANK antagonist is selected from the group consisting of an antibody that specifically binds RANK, an antibody that specifically binds RANKL, osteoprotegerin and an antisense oligonucleotide that blocks translation or transcription of RANK mRNA.

23. A method according to claim 21, wherein the RANK antagonist is a soluble RANK polypeptide that is capable of binding RANKL.

24. The method of claim 21, wherein the soluble RANK polypeptide is encoded by a nucleic acid molecule selected from the group consisting of:
(a) a nucleic acid molecule encoding a polypeptide comprising amino acids x to y of SEQ ID NO: 4, wherein x is selected from the group consisting of amino acids 1 to 33 of SEQ ID NO: 4, and y is selected from the group consisting of amino acids 196 to 213 of SEQ ID NO: 4;
(b) a nucleic acid molecule encoding a polypeptide comprising amino acids x to y of SEQ ID NO: 2, wherein x is selected from the group consisting of amino acids 1 to 35 of SEQ ID NO: 2, and y is selected from the group consisting of amino acids 197 to 214 of SEQ ID NO: 2; and
(c) a nucleic acid molecule capable of hybridizing under stringent conditions with a nucleic acid molecule of (a) or (b) or its complement, wherein the stringent conditions comprise hybridizing in 6×SSC at 63° C., and washing in 3×SSC at 55° C.

25. A method according to claim 21, wherein the soluble RANK polypeptide further comprises a moiety selected from the group consisting of an immunoglobulin Fc domain, a FLAG™ tag, a peptide comprising at least about 6 His residues, a leucine zipper, polyethylene glycol and combinations thereof.

26. A method according to claim 25, wherein the soluble RANK polypeptide comprises amino acids 30-443 of SEQ ID NO: 5.

27. A method of according to claim 21, wherein the patient has a cancer selected from the group consisting of plasmacytoma, monoclonal gammopathy of undetermined significance (MGUS), and Waldenstrom macroglobulinemia.

28. The method of claim 21, wherein the patient has a cancer selected from the group consisting of lung cancer, breast cancer, melanoma, sarcoma, prostate cancer, head and neck cancer, cancer of unknown primary origin, lymphoma, leukemia, kidney cancer, and gastrointestinal cancer.

29. The method of claim 21, wherein the patient has a cancer selected from the group consisting of brain tumor; glioma; neuroblastoma; astrocytoma; medulloblastoma; ependymoma; retinoblastoma; nasopharygeal cancer; basal cell carcinoma; pancreatic cancer; cancer of the bile duct; Kaposi's sarcoma; thynioma; testicular cancer; uterine cancer; vaginal cancer; cervical cancer; ovarian cancer; liver cancer; endometrial cancer; and hemagiopericytoma.

30. The method of claim 21, wherein the patient has a cancer selected from the group consisting of Hodgkin's lymphoma; non-Hodgkin's lymphoma; B-cell acute lymphoblastic leukemia/lymphoma; T-cell acute lymphoblastic leukemia/lymphoma; peripheral T-cell leukemia, adult T-cell leukemia/T-cell lymphoma; NK cell tumor; large granular lymphocytic leukemia; Langerhans cell histiocytosis; myeloid neoplasia; acute myelogenous leukemia; acute promyelocytic leukemia; acute myelomonocytic leukemia; acute monocytic leukemia; a myelodysplastic syndrome; and a chronic myeloproliferative disorder.

31. A method of treating a cancer in a human patient who is not hypercalcemic and whose cancer has not metastasized to bone, the method comprising administering a RANK antagonist to said patient, wherein said patient has a serum calcium level between 9.0 to 10.3 mg/dL if the patient is a man and between 8.9 to 10.2 mg/dL if the patient is a woman, wherein the RANK antagonist is administered in an amount and at a frequency effective to reduce the amount of a surrogate marker that is associated with the patient's cancer.

32. A method according to claim 31, wherein the RANK antagonist is selected from the group consisting of an antibody that specifically binds RANK, an antibody that specifically binds RANKL, osteoprotegerin and an antisense oligonucleotide that blocks translation or transcription of RANK mRNA.

33. A method according to claim 31, wherein the RANK antagonist is a soluble RANK polypeptide that is capable of binding RANKL.

34. The method of claim 31, wherein the soluble RANK polypeptide is encoded by a nucleic acid molecule selected from the group consisting of:
(a) a nucleic acid molecule encoding a polypeptide comprising amino acids x to y of SEQ ID NO: 4, wherein x is selected from the group consisting of amino acids 1 to 33 of SEQ ID NO: 4, and y is selected from the group consisting of amino acids 196 to 213 of SEQ ID NO: 4;
(b) a nucleic acid molecule encoding a polypeptide comprising amino acids x to y of SEQ ID NO: 2, wherein x is selected from the group consisting of amino acids 1 to 35 of SEQ ID NO: 2, and y is selected from the group consisting of amino acids 197 to 214 of SEQ ID NO: 2; and
(c) a nucleic acid molecule capable of hybridizing under stringent conditions with a nucleic acid molecule of (a) or (b) or its complement, wherein the stringent conditions comprise hybridizing in 6×SSC at 63° C., and washing in 3×SSC at 55° C.

35. A method according to claim 34, wherein the soluble RANK polypeptide further comprises a moiety selected from the group consisting of an immunoglobulin Fc domain, a FLAG™ tag, a peptide comprising at least about 6 His residues, a leucine zipper, polyethylene glycol and combinations thereof.

36. A method according to claim 35, wherein the soluble RANK polypeptide comprises amino acids 30-443 of SEQ ID NO: 5.

37. A method of according to claim 31, wherein the patient has a cancer selected from the group consisting of plasmacytoma, monoclonal gammopathy of undetermined significance (MGUS), and Waldenstrom macroglobulinemia.

38. The method of claim 31, wherein the patient has a cancer selected from the group consisting of lung cancer, breast cancer, melanoma, sarcoma, prostate cancer, head and neck cancer, cancer of unknown primary origin, lymphoma, leukemia, kidney cancer, and gastrointestinal cancer.

39. The method of claim 31, wherein the patient has a cancer selected from the group consisting of brain tumor; glioma; neuroblastoma; astrocytoma; medulloblastoma; ependymoma; retinoblastoma; nasopharygeal cancer; basal cell carcinoma; pancreatic cancer; cancer of the bile duct; Kaposi's sarcoma; thymoma; testicular cancer; uterine cancer; vaginal cancer; cervical cancer; ovarian cancer; liver cancer; endometrial cancer; and hemagiopericytoma.

40. The method of claim 31, wherein the patient has a cancer selected from the group consisting of Hodgkin's lymphoma; non-Hodgkin's lymphoma; B-cell acute lymphoblastic leukemia/lymphoma; T-cell acute lymphoblastic leukemia/lymphoma; peripheral T-cell leukemia, adult T-cell leukemia/T-cell lymphoma; NK cell tumor; large granular lymphocytic leukemia; Langerhans cell histiocytosis; myeloid neoplasia; acute myelogenous leukemia; acute promyelocytic leukemia; acute myelomonocytic leukemia; acute monocytic leukemia; a myelodysplastic syndrome; and a chronic myeloproliferative disorder.


SEQUENCE LISTING (6 sequences)

SEQ ID NO: 1 - DNA, 1878 bp, Mus musculus
  CDS (1)..(1875)
atggccccgcgcgcccggcggcgccgccagctgcccgcgccgctgctggcgctctgcgtgctgctcgttccactgcaggtgactctccaggtcactcctccatgcacccaggagaggcattatgagcatctcggacggtgttgcagcagatgcgaaccaggaaagtacctgtcctctaagtgcactcctacctccgacagtgtgtgtctgccctgtggccccgatgagtacttggacacctggaatgaagaagataaatgcttgctgcataaagtctgtgatgcaggcaaggccctggtggcggtggatcctggcaaccacacggccccgcgtcgctgtgcttgcacggctggctaccactggaactcagactgcgagtgctgccgcaggaacacggagtgtgcacctggcttcggagctcagcatcccttgcagctcaacaaggatacggtgtgcacaccctgcctcctgggcttcttctcagatgtcttttcgtccacagacaaatgcaaaccttggaccaactgcaccctccttggaaagctagaagcacaccaggggacaacggaatcagatgtggtctgcagctcttccatgacactgaggagaccacccaaggaggcccaggcttacctgcccagtctcatcgttctgctcctcttcatctctgtggtagtagtggctgccatcatcttcggcgtttactacaggaagggagggaaagcgctgacagctaatttgtggaattgggtcaatgatgcttgcagtagtctaagtggaaataaggagtcctcaggggaccgttgtgctggttcccactcggcaacctccagtcagcaagaagtgtgtgaaggtatcttactaatgactcgggaggagaagatggttccagaagacggtgctggagtctgtgggcctgtgtgtgcggcaggtgggccctgggcagaagtcagagattctaggacgttcacactggtcagcgaggttgagacgcaaggagacctctcgaggaagattcccacagaggatgagtacacggaccggccctcgcagccttcgactggttcactgctcctaatccagcagggaagcaaatctatacccccattccaggagcccctggaagtgggggagaacgacagtttaagccagtgtttcaccgggactgaaagcacggtggattctgagggctgtgacttcactgagcctccgagcagaactgactctatgcccgtgtcccctgaaaagcacctgacaaaagaaatagaaggtgacagttgcctcccctgggtggtcagctccaactcaacagatggctacacaggcagtgggaacactcctggggaggaccatgaaccctttccagggtccctgaaatgtggaccattgccccagtgtgcctacagcatgggctttcccagtgaagcagcagccagcatggcagaggcgggagtacggccccaggacagggctgatgagaggggagcctcagggtccgggagctcccccagtgaccagccacctgcctctgggaacgtgactggaaacagtaactccacgttcatctctagcgggcaggtgatgaacttcaagggtgacatcatcgtggtgtatgtcagccagacctcgcaggagggcccgggttccgcagagcccgagtcggagcccgtgggccgccctgtgcaggaggagacgctggcacacagagactcctttgcgggcaccgcgccgcgcttccccgacgtctgtgccaccggggctgggctgcaggagcagggggcaccccggcagaaggacgggacatcgcggccggtgcaggagcagggtggggcgcagacttcactccatacccaggggtccggacaatgtgcagaatga

SEQ ID NO: 2 - protein, 625 aa, Mus musculus
MAPRARRRRQLPAPLLALCVLLVPLQVTLQVTPPCTQERHYEHLGRCCSRCEPGKYLSSKCTPTSDSVCLPCGPDEYLDTWNEEDKCLLHKVCDAGKALVAVDPGNHTAPRRCACTAGYHWNSDCECCRRNTECAPGFGAQHPLQLNKDTVCTPCLLGFFSDVFSSTDKCKPWTNCTLLGKLEAHQGTTESDVVCSSSMTLRRPPKEAQAYLPSLIVLLLFISVVVVAAIIFGVYYRKGGKALTANLWNWVNDACSSLSGNKESSGDRCAGSHSATSSQQEVCEGILLMTREEKMVPEDGAGVCGPVCAAGGPWAEVRDSRTFTLVSEVETQGDLSRKIPTEDEYTDRPSQPSTGSLLLIQQGSKSIPPFQEPLEVGENDSLSQCFTGTESTVDSEGCDFTEPPSRTDSMPVSPEKHLTKEIEGDSCLPWVVSSNSTDGYTGSGNTPGEDHEPFPGSLKCGPLPQCAYSMGFPSEAAASMAEAGVRPQDRADERGASGSGSSPSDQPPASGNVTGNSNSTFISSGQVMNFKGDIIVVYVSQTSQEGPGSAEPESEPVGRPVQEETLAHRDSFAGTAPRFPDVCATGAGLQEQGAPRQKDGTSRPVQEQGGAQTSLHTQGSGQCAE

SEQ ID NO: 3 - DNA, 1851 bp, Homo sapiens
  CDS (1)..(1851)
atggccccgcgcgcccggcggcgccgcccgctgttcgcgctgctgctgctctgcgcgctgctcgcccggctgcaggtggctttgcagatcgctcctccatgtaccagtgagaagcattatgagcatctgggacggtgctgtaacaaatgtgaaccaggaaagtacatgtcttctaaatgcactactacctctgacagtgtatgtctgccctgtggcccggatgaatacttggatagctggaatgaagaagataaatgcttgctgcataaagtttgtgatacaggcaaggccctggtggccgtggtcgccggcaacagcacgaccccccggcgctgcgcgtgcacggctgggtaccactggagccaggactgcgagtgctgccgccgcaacaccgagtgcgcgccgggcctgggcgcccagcacccgttgcagctcaacaaggacacagtgtgcaaaccttgccttgcaggctacttctctgatgccttttcctccacggacaaatgcagaccctggaccaactgtaccttccttggaaagagagtagaacatcatgggacagagaaatccgatgcggtttgcagttcttctctgccagctagaaaaccaccaaatgaaccccatgtttacttgcccggtttaataattctgcttctcttcgcgtctgtggccctggtggctgccatcatctttggcgtttgctataggaaaaaagggaaagcactcacagctaatttgtggcactggatcaatgaggcttgtggccgcctaagtggagataaggagtcctcaggtgacagttgtgtcagtacacacacggcaaactttggtcagcagggagcatgtgaaggtgtcttactgctgactctggaggagaagacatttccagaagatatgtgctacccagatcaaggtggtgtctgtcagggcacgtgtgtaggaggtggtccctacgcacaaggcgaagatgccaggatgctctcattggtcagcaagaccgagatagaggaagacagcttcagacagatgcccacagaagatgaatacatggacaggccctcccagcccacagaccagttactgttcctcactgagcctggaagcaaatccacacctcctttctctgaacccctggaggtgggggagaatgacagtttaagccagtgcttcacggggacacagagcacagtgggttcagaaagctgcaactgcactgagcccctgtgcaggactgattggactcccatgtcctctgaaaactacttgcaaaaagaggtggacagtggccattgcccgcactgggcagccagccccagccccaactgggcagatgtctgcacaggctgccggaaccctcctggggaggactgtgaacccctcgtgggttccccaaaacgtggacccttgccccagtgcgcctatggcatgggccttccccctgaagaagaagccagcaggacggaggccagagaccagcccgaggatggggctgatgggaggctcccaagctcagcgagggcaggtgccgggtctggaagctcccctggtggccagtcccctgcatctggaaatgtgactggaaacagtaactccacgttcatctccagcgggcaggtgatgaacttcaagggcgacatcatcgtggtctacgtcagccagacctcgcaggagggcgcggcggcggctgcggagcccatgggccgcccggtgcaggaggagaccctggcgcgccgagactccttcgcggggaacggcccgcgcttcccggacccgtgcggcggccccgaggggctgcgggagccggagaaggcctcgaggccggtgcaggagcaaggcggggccaaggcttga

SEQ ID NO: 4 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 5 - protein, 443 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGRSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 6 aa, Artificial Sequence
  synthetic peptide capable of binding metals that is useful as an affinity tag for proteins
HHHHHH